(19) United States
(12) Patent Application Publication
(10) Pub. No.: US 2005/0002983 A1
(43) Pub. Date: Jan. 6, 2005

(54) Devices, methods, and compositions to prevent restenosis

(76) Inventor: Robert G. Johnson JR., Lafayette, CA (US)

Correspondence Address: Gary W. Ashley;Kosan Biosciences, Inc., 3832 Bay Center Place, Hayward, CA, 94545, US

(21) Appl. No.: 10/814,490

(22) Filed: Mar. 29, 2004

Related U.S. Application Data
(60) Provisional application No. 60/458,521, filed on Mar. 28, 2003.
(60) Provisional application No. 60/490,098, filed on Jul. 24, 2003.

(51) Int. Cl. A61K 31/427; A61K 31/33

(52) U.S. Cl. 424/423; 514/183; 514/365

(57) ABSTRACT

Medical devices, compositions and methods to prevent or reduce restenosis using epothilones, geldanamycin analogs, and rapamycin or rapamycin analogs alone or in synergistic combinations.

### CROSS-REFERENCE TO RELATED APPLICATIONS

This application claims priority under 35 U.S.C. §119(e) to U.S. Provisional Patent Application Ser. Nos. 60/458,521, filed 28 Mar. 2003, and 60/490,098, filed 24 Jul. 2003; each of which is incorporated herein by reference in its entirety.

### FIELD OF THE INVENTION

The invention relates to compositions, methods, and devices to reduce or eliminate restenosis. The invention is thus relevant to the areas of pharmacology, medicine, especially cardiovascular medicine, and medical devices.

### BACKGROUND OF THE INVENTION

Atherosclerosis is the formation of a hardened plaque comprising cholesterol, fatty acids, cellular wastes, and calcium along the walls of medium and large arteries. Such plaques can cause a narrowing (“stenosis”) of a blood vessel, such as a medium or large artery, and is a leading cause of heart attack and stroke. Typically, atherosclerosis is treated using balloon angioplasty (also called Percutaneous Transluminal Coronary Angioplasty or “PTCA”) in which a catheter is inserted in a major artery of the patient and is guided to a major artery of the heart. A balloon located in the distal end of the catheter is inflated to push the plaque against the wall of the constricted vessel, thus widening the vessel and improving blood flow. More recently, small metallic spring-like devices called stents can be inserted at the point of construction to provide a supporting framework that maintains the shape of the vessel.

Unfortunately, these procedures do not always provide permanent solutions. In about 40% of all PTCA procedures and about 25% of stentings, the stenosis recurs within about six months of the procedure. Such recurrence is called “restenosis”, and, in the case of restenosis following stent insertion (“in-stent restenosis”). In-stent restenosis occurs when scar tissue grows under the layer of otherwise healthy vessel tissue that grows over the framework of the stent and provides improved blood flow through the stent to a degree sufficient to restrict blood flow through the stented segment of the vessel.

Recently, specially coated drug-eluting stents that include a cytotoxic agent have been provided to reduce the occurrence of in-stent restenosis. A variety of drugs have been used in such stents, including sirolimus (rapamycin), which inhibits growth of smooth muscle cells (“SMCs”), paclitaxel, an antiproliferative agent, and several anti-inflammatory drugs. See, for example: Ozaki et al. (1996), “New stent technologies,” Prog. Cardiovasc. Disease 39(2): 129-40; Lincoff et al. (1997) “Sustained local delivery of dexamethasone by a novel intravascular eluting stent to prevent restenosis in the porcine coronary injury model.” Journal of the American College of Cardiology 29(4): 808-816; Violaris et al. (1997) “Endovascular stents: a ‘break through technology’, future challenges.” Int J Card Imaging 13(1): 3-13; Garas et al. (2001) “Overview of therapies for prevention of restenosis after coronary interventions.” Pharmacology & Therapeutics 92(2-3): 165-178; Garas et al. (2001) “Overview of therapies for prevention of restenosis after coronary interventions.” Pharmacol Ther 92(2-3): 165-78; Regar et al. (2001) “Stent development and local drug delivery.” Br Med Bull 59: 22748; Chieffo & Colombo (2002) “Drug-eluting stents.” Minerva Cardioangiol 50(5): 419-29; Greenberg & Cohen (2002) “Examining the economic impact of restenosis: implications for the cost-effectiveness of an antiproliferative stent.” Z Kardiol 91 Suppl 3: 137-43; Grube & Bullesfeld (2002) “Initial experience with paclitaxel-coated stents.” J Interv Cardiol 15(6): 471-5; Grube et al. (2002) “Drug eluting stents: initial experiences.” Z Kardiol 91 Suppl 3: 44-8; Hehrlein et al. (2002) “Drug-eluting stent: the “magic bullet” for prevention of restenosis?” Basic Res Cardiol 97(6): 417-23; Liistro et al. (2002) “First clinical experience with a paclitaxel derivate-eluting polymer stent system implantation for in-stent restenosis: immediate and long-term clinical and angiographic outcome.” Circulation 105(16): 1883-6; Muller et al. (2002) “[State of treatment of coronary artery disease by drug releasing stents].” Herz 27(6): 508-13; Peters (2002) “Can angiotensin receptor antagonists prevent restenosis after stent placement?” American Journal of Cardiovascular Drugs 2(3): 143-148; Prebitero and Asioli (2002) “[Drug-eluting stents do they make the difference?].” Minerva Cardioangiol 50(5): 431-42; Sheiban et al. (2002) “Drug-eluting stent: the emerging technique for the prevention of restenosis.” Minerva Cardioangiol 50(5): 443-53; Fattori & Piva (2003) “Drug-eluting stents in vascular intervention.” Lancet 361(9353): 247-9. In particular, U.S. Pat. No. 6,231,600 to Zhong describes a hybrid stent coating including a non-thrombogenic agent and paclitaxel-containing polymer that allows time-release of the paclitaxel to reduce or prevent in-stent restenosis. U.S. patent application No. 20030207856 discloses stents coated with the Hsp90 inhibitor geldanamycin.

Nevertheless, it would be advantageous to provide additional drug-eluting stents having different restenosis-preventing or reducing agents. For example, paclitaxel has such great cytotoxicity that necrosis of the vessel wall has been observed. Thus, paclitaxel has relatively narrow therapeutic window that can complicate formulation and administration.

### SUMMARY OF THE INVENTION

The present invention addresses these needs by providing compositions, methods, and devices that substantially reduce or prevent restenosis. We have unexpectedly found that certain geldanamycin analogs, particularly the 17-amino-17-desmethoxy-geldanamycins such as 17-allylamino-17-desmethoxygeldanamycin (17-AAG) and 17-(dimethylaminoethyl)-17-desmethoxygeldanamycin (DMAG), display selective cytotoxicity against smooth muscle cells and hence provide unique advantages for use in controlling restenosis. Further, we have discovered that particular combinations of cytotoxic drugs are unexpectedly synergistic, thus reducing the concentrations of the individual cytotoxic drugs needed to prevent restenosis.

In one aspect, the present invention includes a medical device configured to deliver one or more drugs described herein to a blood vessel to reduce the degree or substantially prevent the occurrence of restenosis in the blood vessel. In one embodiment, the drug is an epothilone. In another embodiment, the drug is a geldanamycin derivative. In still another embodiment, the drug is a rapamycin analog. In a more particular embodiment, the drug is a desoxyepothilone, and, more particularly, epothilone D. In another embodiment, the drug is 17-allylamino-17-desmethoxygeldanamycin, 17-[2-(dimethylamino)ethylamino]-17-desmethoxygeldanamycin, 17-[2-(dimethylamino)ethylamino]-17-desmethoxy-11-O-methylgeldanamycin. In yet another embodiment, the drug is 17-azetidinyl-17-desmethoxy-geldanamycin. In some embodiments, the above-described drugs are used in combination to provide a synergistic effect. In some embodiments, the drug or drugs described herein is further combined with an anti-inflammatory. In some embodiments, the device is a stent. In other embodiments, the device is a polymer wrapper or device used to cover vascular anastomoses. In some embodiments, the device includes at least one coating effective to deliver one or more drugs described herein to a blood vessel.

In another aspect, the present invention provides compositions to reduce the degree or substantially prevent the occurrence of restenosis in the blood vessel. In one embodiment, the drug is an epothilone. In another embodiment, the drug is geldanamycin or a geldanamycin derivative. In still another embodiment, the drug is a rapamycin analog. In a more particular embodiment, the drug is a desoxyepothilone, and, more particularly, epothilone D. In another embodiment, the drug is 17-allylamino-17-desmethoxygeldanamycin, 17-[2-(dimethylamino)ethylamino]-17-desmethoxy-geldanamycin, or 17-[2-(dimethylamino)ethylamino]-17-desmethoxy-11-O-methylgeldanamycin. In yet another embodiment, the drug is 17-azetidinyl-17-desmethoxygeldanamycin. In some embodiments, the drug or drugs described herein is further combined with an anti-inflammatory agent. The composition can include a polymer such that the drug of the invention elutes from the polymer into blood vessel tissues proximal to the polymer

In still another aspect, the present invention provides methods to to reduce the degree or substantially prevent the occurrence of restenosis in the blood vessel. In one embodiment, the method of the invention includes delivering a drug described herein to a blood vessel requiring treatment for, or prevention of, restenosis, in an amount sufficient to substantially reduce, or substantially prevent, restenosis in such blood vessel. In one embodiment, the drug is an epothilone. In another embodiment, the drug is geldanamycin or a geldanamycin derivative. In still another embodiment, the drug is a rapamycin analog. In a more particular embodiment, the drug is a desoxyepothilone, and, more particularly, epothilone D. In another embodiment, the drug is 17-allylamino-17-desmethoxygeldanamycin, 17-[2-(dimethylamino)ethylamino]-17-desmethoxy-geldanamycin, or 17-[2-(dimethylamino)ethylamino]-17-desmethoxy-11-O-methylgeldanamycin. In yet another embodiment, the drug is 17-azetidinyl-17-desmethoxygeldanamycin. In some embodiments, the drug or drugs described herein is further combined with an anti-inflammatory agent.

These and other aspects and advantages will become apparent when the Description below is read in conjunction with the accompanying Drawings.

### BRIEF DESCRIPTION OF THE DRAWINGS

FIG. 1A and FIG. 1B are plots of cell viability for smooth muscle cells (“SMC”, FIG. 1A) and human umbilical vein endothelial cells (“HUVEC”, FIG. 1B) exposed to 17-allylaminogeldanamycin (“17-AAG”) as measured by optical density using the methods described in Example 1 herein. The SMC and HUVEC were exposed to a control (♦) and to 17-AAG at concentrations of 10 nanomolar (“nM”,▪), 100 nM (▴), and 1,000 nM (x).

FIG. 2A and FIG. 2B are plots of cell viability for smooth muscle cells (“SMC”, FIG. 2A) and human umbilical vein endothelial cells (“HUVEC”, FIG. 2B) exposed to 17-[(2-dimethylamino)ethylamino]geldanamycin (“17-DMAG”) as measured by optical density using the methods described herein. The SMC and HUVEC were exposed to a control (♦) and to 17-DMAG at concentrations of 10 nanomolar (“nM”,▪), 100 nM (▴), and 1,000 nM (x).

FIG. 3A and FIG. 3B are plots of cell viability for smooth muscle cells (“SMC”, FIG. 3A) and human umbilical vein endothelial cells (“HUVEC”, FIG. 3B) exposed to KOS-862 (epothilone D) as measured by optical density using the methods described herein. The SMC and HUVEC were exposed to a control (♦) and to epothilone D at concentrations of 10 nanomolar (“nM”,▪), 100 nM (Δ), and 1,000 nM (x).

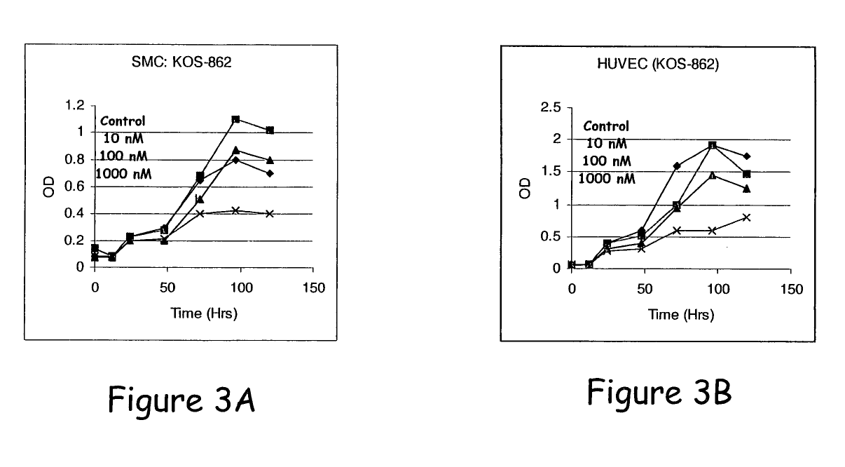

FIG. 4 is a plot of the Combination Index for the combination of rapamycin and 17-AAG in SMC, which indicates synergistic effect.

FIG. 5 is a plot of the Combination Index for the combination of rapamycin and KOS-862 in SMC, which indicates synergistic effect.

FIG. 6A and FIG. 6B are plots demonstrating the synergistic effect of combining 17-AAG with rapamycin. FIG. 6A shows the change in viability of DLD-1 cells as measured by optical density (“OD”) for rapamycin (solid line), 17-AAG (squares), and their combination (diamonds) at concentrations of 0 to 120 nM. FIG. 6B shows the Combination Index for the combination of rapamycin and 17-AAG, which indicates synergistic effect.

FIG. 7A and FIG. 7B are plots demonstrating the synergistic effect of combining 17-AAG with rapamycin. FIG. 7A shows the change in viability of DLD-1 cells as measured by optical density (“OD”) for rapamycin (solid line), KOS-862 (epothilone D) (squares), and their combination (diamonds) at concentrations of 0 to 120 nM. FIG. 7B shows the Combination Index for the combination of rapamycin and KOS-862, which indicates a synergistic effect.

FIG. 8 shows release kinetics for epothilone D (“KOS-862”) from various polymer matrices. Epothilone D is released from poly(lactide) (▴) at a rate of approximately 6 micrograms/day and from polyurethane (♦) at 1.58 micrograms/day.

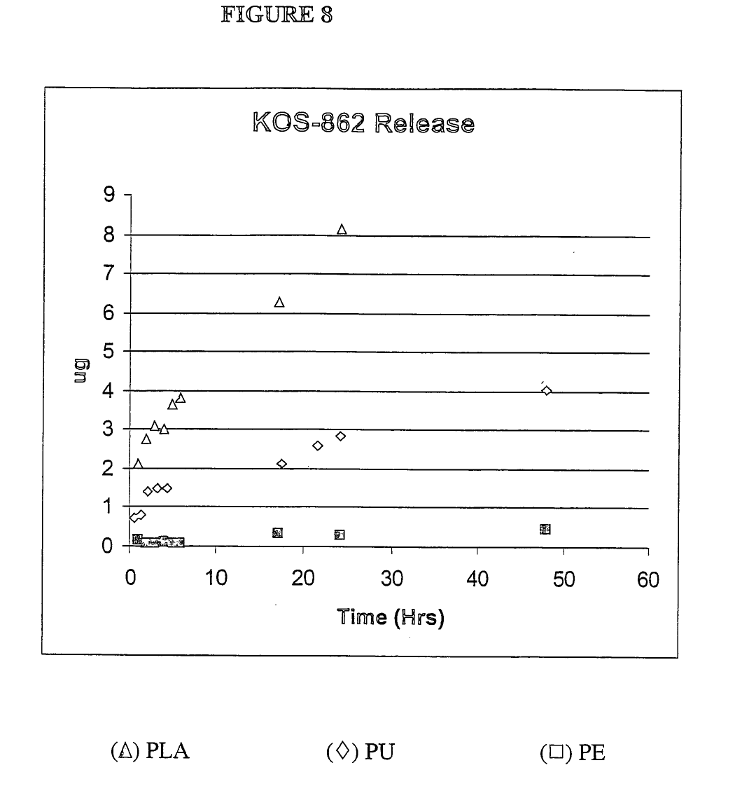

### DETAILED DESCRIPTION OF THE INVENTION

In one embodiment, the present invention provides stents including a coating that releases a drug selected from the group of epothilones and geldanamycins. Suitable epothilones for combination in the present invention can be any epothilone, and, more particularly, any epothilone having useful therapeutic properties; see, for example, Hoefle et al. (1993) Ger. Offen. DE 4138042; Nicolaou et al. (1998) PCT Publication WO 98/25929; Reichenbach et al. (1998) PCT Publication WO 98/22461; Danishefsky et al. (1999) PCT Publication WO 99/01124; Hoefle et al. (1999) PCT Publication WO 99/65913; Nicolaou et al. (1999) PCT Publication WO 99/67253; Nicolaou (1999) PCT Publication WO 99/67252; Vite et al. (1999) PCT Publication WO 99/54330; Vite et al. (1999) PCT Publication WO 99/02514; Vite et al. (1999) PCT Publication WO 99/54319; Hoefle et al. (2000) Ger. Offen. DE 19907588; Hoefle et al. (2000) PCT Publication WO 00/50423; Danishefsky et al. (2001) U.S. Pat. No. 6,204,388; Danishefsky et al. (2001) PCT Publication WO 01/64650; Santi et al. (2001) PCT Publication WO 01/92255; Avery (2002) PCT Publication WO 02/30356; Danishefsky et al. (2002) U.S. patent application Publ. No. 20020058286; Nicolaou et al. (2002) U.S. Pat. No. 6,441,186; Nicolaou et al. (2002) U.S. Pat. No. 6,380,394; Wessjohan & Scheid (2002) Ger. Offen. DE 10051136; and White et al. (2002) U.S. patent application Publ. No. 20020062030; each of which is incorporated herein by reference for all purposes. Such epothilones can be obtained using any combination of total chemical synthesis, partial chemical synthesis, or chemobiosynthesis methods and materials known to those of skill in organic chemistry, medicinal chemistry, and biotechnology arts. Specific examples of epothilones having useful therapeutic properties include, but are not limited to, epothilone A, epothilone B, epothilone C, epothilone D, 4-desmethylepothilone D, azaepothilone B (epothilone B lactam), 21-aminoepothilone B, 9, 10-dehydroepothilone D, 9, 10-dehydro-26-trifluoroepothilone D, 11-hydroxyepothilone D, 19-oxazolylepothilone D, 10, 11-dehydroepothilone D, 19-oxazolyl-10, 11-dehydroepothilone D, and trans-9,10-dehydroepothilone D.

In another embodiment, the drug is geldanamycin or an analog or derivative thereof. In one embodiment, the drug is geldanamycin. In preferred embodiments, the drug is an analog of geldanamycin, for example a 17-(substituted amino)-17-desmethoxygeldanamycin. In one preferred embodiment, the drug is 17-allylamino-17-desmethoxygeldanamycin (“17-AAG”). In still another embodiment, the drug is 17-[2-(dimethylamino)ethylamino]-17-desmethoxygeldanamycin (“17-DMAG”). In another embodiment, the drug is 17-[2-(dimethylamino)ethylamino]-17-desmethoxy-11-O-methylgeldanamycin. In yet another embodiment, the drug is 17-azetidinyl-17-desmethoxygeldanamycin. These compounds can be obtained using methods known to those having skill in the organic and medicinal chemistry arts; see, for example, Sasaki et al. (1981) U.S. Pat. No. 4,261,989; Schnur et al. (1999) U.S. Pat. No. 5,932,566; Zhang et al. (2003) PCT Publication WO 03/026571; Santi et al. (2003) PCT Publication WO 03/13430, as well as in co-pending U.S. patent applications Ser. Nos. 60/389,225; 60/393,929; 60/395,275; 60/415,326; and 60/420,820. Each of the foregoing U.S. patent applications is incorporated herein by reference for all purposes.

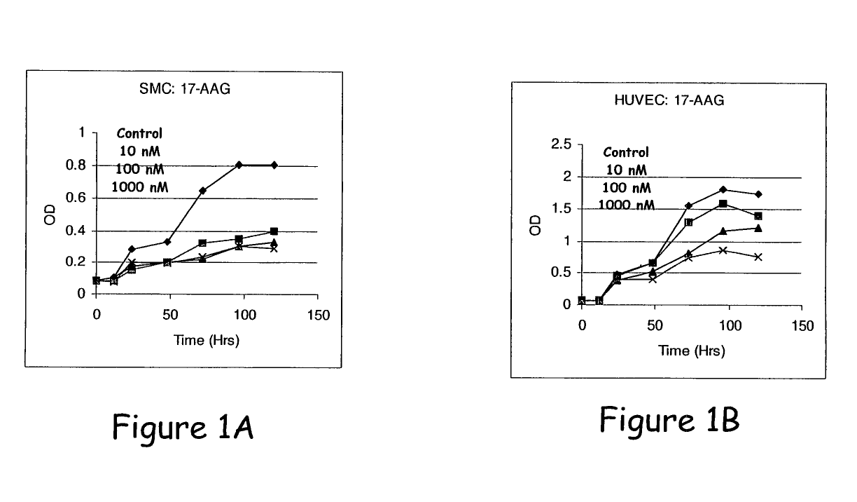

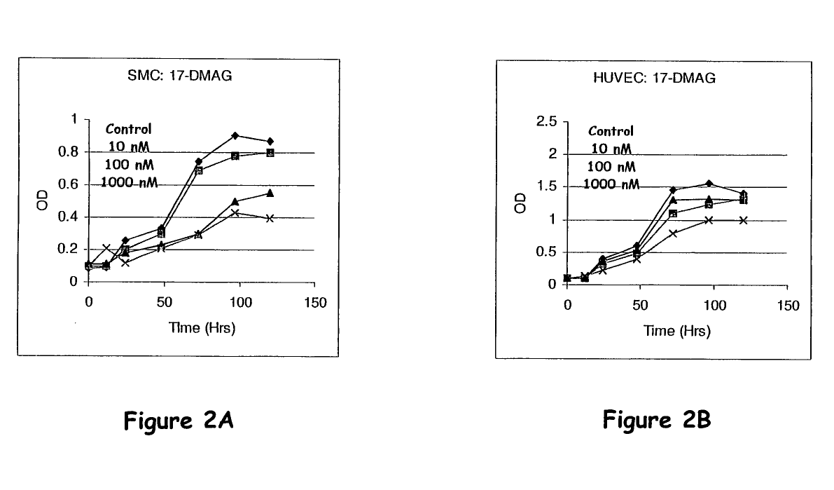

While geldanamycin itself is a potent cytotoxin, with IC₅₀ values for smooth muscle cells of approximately 0.9 nM, such high cytotoxicity may be problematic for the treatment of restenosis where the localized drug concentrations can be high. For effective treatment of restenosis, a drug showing selective cytotoxicity against smooth muscle cells over endothelial cells, for example, would allow treatment of restenosis with minimal damage to other cell types not involved in restenosis. We have unexpectedly found that certain geldanamycin analogs, particularly the 17-amino-17-desmethoxy-geldanamycins such as 17-allylamino-17-desmethoxygeldanamycin (17-AAG) and 17-(dimethylaminoethyl)-17-desmethoxygeldanamycin (DMAG), display selective cytotoxicity against smooth muscle cells (see FIGS. 1 and 2). While these analogs are generally less cytotoxic than geldanamycin itself, 17-AAG for example shows an IC₅₀ of about 10 nM against smooth muscle cells, they show substantially higher IC₅₀ values against endothelial cells. Thus, these analogs offer unexpected advantages over geldanamycin itself in the treatment of restenosis.

In another embodiment of the invention, the drug is rapamycin or a rapamycin analog. By “rapamycin or a rapamycin analog” is meant a compound of structure (I), 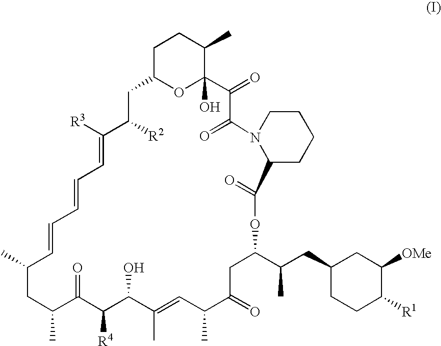
wherein R¹ is hydroxy, alkoxy, hydroxyethoxy, aryloxy, or heteroaryl; R² is H or OMe; R³ is H or Me; and R⁴ is H, OH, or OMe. Specific examples of rapamycin analogs are described in PCT Publication WO 01/38416, which is incorporated herein by reference for all purposes.

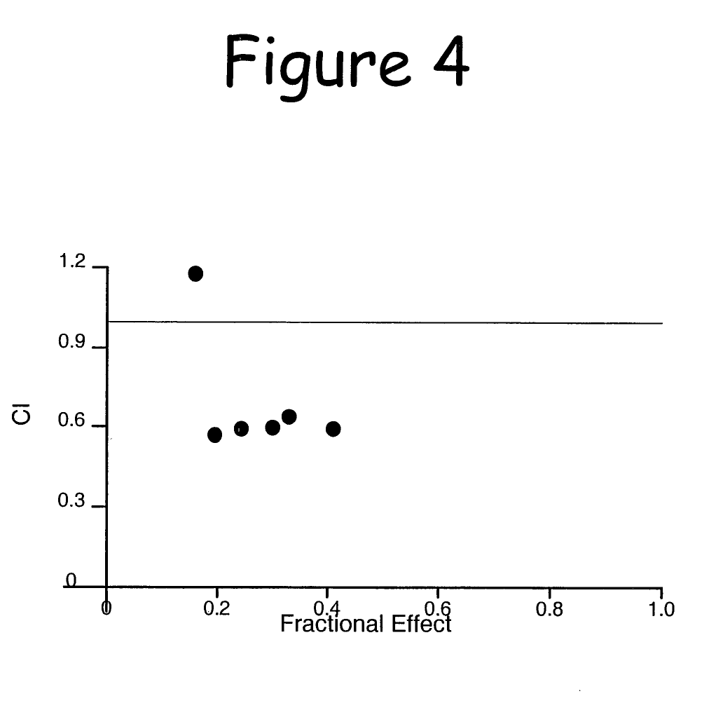

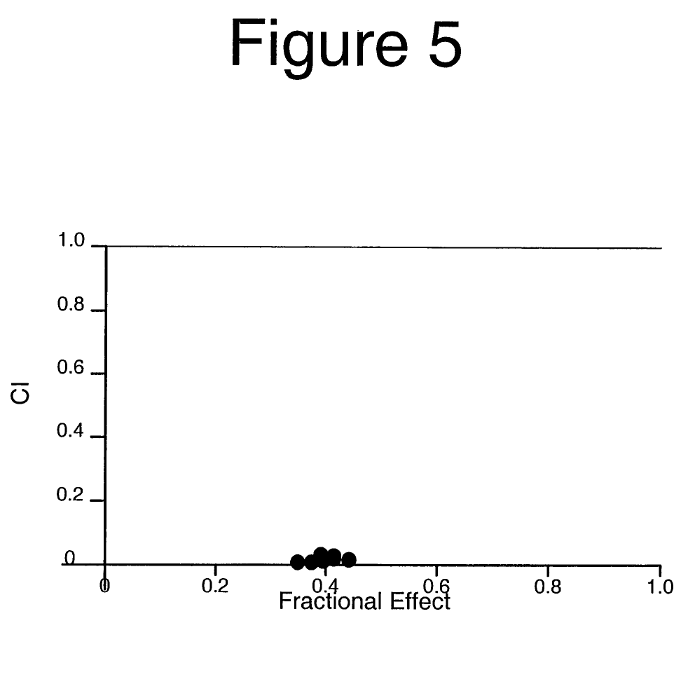

In preferred embodiments, rapamycin or a rapamycin analog is administered in combination with a second drug to provide a synergistic cytotoxic effect on smooth muscle cells. Examples of synergistic combinations include rapamycin with a geldanamycin analog, as illustrated for rapamycin and 17-AAG in FIG. 4, and rapamycin with epothilone D, as demonstrated in FIG. 5. The use of synergistic mixtures is highly advantageous, as it allows use of lower drug loadings and/or increased effectiveness at preventing restenosis. The ratios of the two drugs may be determined by methods known in the art, for example as described below in Example 2.

In some embodiments, the drug or drug combination is combined with a stent so that the process of restenosis is substantially mitigated or prevented. Such stents may be metallic or made of a bioresorbable polymer. Examples of stents suitable with the present invention include, but are not limited to, stents configured to elute a drug as are known to those of skill in the cardiovascular medicine and medical device arts. See, for example, Aggarwal et al. (1996) “Antithrombotic potential of polymer-coated stents eluting platelet glycoprotein IIb/IIIa receptor antibody.” Circulation 94(12): 3311-3317; Ozaki et al. (1996), “New stent technologies,” Prog. Cardiovasc. Disease 39(2): 129-40; Lincoff et al. (1997) “Sustained local delivery of dexamethasone by a novel intravascular eluting stent to prevent restenosis in the porcine coronary injury model.” Journal of the American College of Cardiology 29(4): 808-816; Violaris et al. (1997) “Endovascular stents: a ‘break through technology’, future challenges.” Int J Card Imaging 13(1): 3-13; Garas et al. (2001) “Overview of therapies for prevention of restenosis after coronary interventions.” Pharmacology & Therapeutics 92(2-3): 165-178; Garas et al. (2001) “Overview of therapies for prevention of restenosis after coronary interventions.” Pharmacol Ther 92(2-3): 165-78; Regar et al. (2001) “Stent development and local drug delivery.” Br Med Bull 59: 227-48; Chieffo & Colombo (2002) “Drug-eluting stents.” Minerva Cardioangiol 50(5): 419-29; Greenberg & Cohen (2002) “Examining the economic impact of restenosis: implications for the cost-effectiveness of an antiproliferative stent.” Z Kardiol 91 Suppl 3: 137-43; Grube & Bullesfeld (2002) “Initial experience with paclitaxel-coated stents.” J Interv Cardiol 15(6): 471-5; Grube et al. (2002) “Drug eluting stents: initial experiences.” Z Kardiol 91 Suppl 3: 44-8; Hehrlein et al. (2002) “Drug-eluting stent: the “magic bullet” for prevention of restenosis?” Basic Res Cardiol 97(6): 417-23; Liistro et al. (2002) “First clinical experience with a paclitaxel derivate-eluting polymer stent system implantation for in-stent restenosis: immediate and long-term clinical and angiographic outcome.” Circulation 105(16): 1883-6; Muller et al. (2002) “[State of treatment of coronary artery disease by drug releasing stents].” Herz 27(6): 508-13; Peters (2002) “Can angiotensin receptor antagonists prevent restenosis after stent placement?” American Journal of Cardiovascular Drugs 2(3): 143-148; Prebitero and Asioli (2002) “[Drug-eluting stents do they make the difference?].” Minerva Cardioangiol 50(5): 431-42; Sheiban et al. (2002) “Drug-eluting stent: the emerging technique for the prevention of restenosis.” Minerva Cardioangiol 50(5): 443-53; Fattori & Piva (2003) “Drug-eluting stents in vascular intervention.” Lancet 361(9353): 247-9; Klugherz et al. (2000) “Gene delivery from a DNA controlled-release stent in porcine coronary arteries.” Nature Biotechnology 18(11): 1181-1184; Carlyle et al. (2002) Eur. Pat. Appl. Ep 1236478; Farb et al. (2002) “Oral Everolim Inhibits In-Stent Neointimal Growth.” Circulation 106(18): 2379-2384; Morice et al. (2002) “A randomized comparison of a sirolim-eluting stent with a standard stent for coronary revascularization.” New England Journal of Medicine 346(23): 1773-1780; Moses et al. (2002) “Perspectives of drug-eluting stents. The next revolution.” American Journal of Cardiovascular Drugs 2(3): 163-172; Shah et al. (2002) “Background Incidence of Late Malapposition After Bare-Metal Stent Implantation.” Circulation 106(14): 1753-1755; Swanson et al. (2002) “Human internal mammary artery organ culture model of coronary stenting: a novel investigation of smooth mcle cell response to drug-eluting stents.” Clinical Science 103(4): 347-353; Virmani et al. (2002) “Mechanism of Late In-Stent Restenosis After Implantation of a Paclitaxel Derivate-Eluting Polymer Stent System in Humans.” Circulation 106(21): 2649-2651; and Yoon et al. (2002) “Local delivery of nitric oxide from an eluting stent to inhibit neointimal thickening in a porcine coronary injury model.” Yonsei Medical Journal 43(2): 242-251; each of which is incorporated herein by reference for all purposes.

In other embodiments, the stent is coated with one or more polymer substances to facilitate blood flow over the stent surfaces and to provide a reservoir of the drug such that the drug is released to provide substantial mitigation or prevention of restenosis. Examples of such polymer are known to those of skill in the cardiovascular medicine and medical device arts; see, for example, Levy et al. (1994) “Strategies for treating arterial restenosis using polymeric controlled release implants.” Biotechnol. Bioact. Polym., [Proc. Am. Chem. Soc. Symp.]: 259-68; De Scheerder et al. (1995) “Biocompatibility of polymer-coated oversized metallic stents implanted in normal porcine coronary arteries.” Atherosclerosis (Shannon, Ireland) 114(1): 105-14; Peng et al. (1996) “Role of polymers in improving the results of stenting in coronary arteries.” Biomaterials 17(7): 685-94; Tartaglia et al. (1996) Can. Pat. Appl. Ca 2164684; Herdeg et al. (1998) “Antiproliferative stent coatings: Taxol and related compounds.” Semin Interv Cardiol 3(3-4): 197-9; Reich et al. (1998) PCT Publication WO 98/08884; Santos et al. (1998) “Local administration of L-703081 using a composite polymeric stent reduces platelet deposition in canine coronary arteries.” American Journal of Cardiology 82(5): 673-675; Whitbourne (1998) PCT Publication WO 98/32474; Tsuji et al. (2003) “Biodegradable stents as a platform to drug loading,” Int J Cardiovasc Intervent. 5(1):13-6; Lahann et al. (1999) “Improvement of hemocompatibility of metallic stents by polymer coating.” Journal of Materials Science: Materials in Medicine 10(7): 443-448; Piro et al. (1999) “An electrochemical method for entrapment of oligonucleotides into a polymer-coated electrode.” Proceedings of the International Symposium on Controlled Release of Bioactive Materials 26th: 1176-1177; Bar et al. (2000) “New biocompatible polymer surface coating for stents results in a low neointimal response.” Journal of Biomedical Materials Research 52(1): 193-198; Le More et al. (2000) Fr. Demande FR 2785812; Verweire et al. (2000) “Evaluation of fluorinated polymers as coronary stent coating.” Journal of Materials Science: Materials in Medicine 11(4): 207-212; Zhong (2001) U.S. Pat. No. 6,231,600; Heublein et al. (2002) Polymerized degradable hyaluronan-a platform for stent coating with inherent inhibitory effects on neointimal formation in a porcine coronary model.” Int J Artif Organs 25(12): 1166-73; Lewis et al. (2002) “Analysis of a phosphorylcholine-based polymer coating on a coronary stent pre- and post-implantation.” Biomaterials 23(7): 1697-1706; Roorda et al. (2002) PCT Publication WO 02/94335; and Rosenblum et al. (2003) PCT Publication WO 03/07785; each of which is incorporated herein by reference for all purposes.

In preferred embodiments, the polymer is selected from the group consisting of poly(ester-amides) (“PEA”), polylactides (“PLA”), and amino acid-based polyurethanes (“PU”). Suitable poly(ester-amides) are described in Lee et al. (2002) “In-vivo biocompatibility evaluation of stents coated with a new biodegradable elastomeric and functional polymer,” Coron Artery Dis. 2002 Jun;13(4):237-41; and U.S. Pat. No. 6,703,040, which is incorporated herein by reference, and are prepared by synthesizing monomers of two alpha amino acids with diols and diacids. In preferred embodiments, the poly(ester-amide) is prepared from L-leucine, L-lysine, hexanediol, and sebacic acid. The drugs can be chemically deposited into the polymer matrix or conjugated onto the polymer backbone via the carboxyl groups of the L-lysine. The polymer is elastomeric and can be crosslinked in situ using photo activators, resulting in a strong yet biocompatible and reabsorbable polymer. The polylactide-based polymers can be made from L-lactide, caprolactone, and polyethylene glycol monomers in varying ratios. The polyurethane polymers can be made by condensing monomers of alpha amino acids, such as L-leucine and L-lysine, with a diol. The carboxyl groups of lateral L-lysine on the polymer can be used as an attachment site for coupling drugs. The polyurethane polymers generally show a faster degradation rate than the poly(ester-amide) polymers, and are generally similar in terms of biocompatibility and reabsorbability.

When used for coating medical devices, for example stents, solutions of the polymer and drug in volatile solvents, either individually or in combination, may be applied to the surface by spraying or by dipping. The volatile solvents are then allowed to evaporate, resulting in a coating on the device comprising the polymer and the drug. Varying proportions of polymer and drug may be applied, depending upon the potency of the drug and the time period over which the drug is to be released from the medical device. To further control the rate of release of the drug, a topcoat of additional polymer may be applied to the coated device. The medical devices may subsequently be rendered aseptic, for example by gamma irradiation.

In another embodiment, the drug or drugs described herein can be used with a medical device to prevent restenosis after vascular anastomosis, for example by being combined with a polymer sheath or wrapping around the vessel wall. Such materials are available commercially from Secant Medical, LLC of Perkasie, Pa., USA. Further examples of suitable devices that may be coated with the compositions of the invention may be found, for example in U.S. Pat. No. 6,371,965, which is incorporated herein by reference. These devices may be useful particularly after vascular anastomosis such as occurs during coronary artery bypass graft surgery.

In other embodiments, one or more anti-inflammatory drugs effective to reduce or prevent inflammatory processes from occurring in the vessel wall is included with the drug or drugs described herein above. Examples of suitable anti-inflammatory drugs include, but are not limited to, rapamycin and rapamycin analogs described in WO 01/38416, which is incorporated herein by reference for all purposes.

In other embodiments, one or more of the drugs described above are deposited directly to the site of restenosis. Deposition can be accomplished using, e.g., a catheter or suitable drug delivery device.

### EXAMPLES

The following Examples are provided to illustrate certain aspects of the present invention and to aid those of skill in the art in the art in practicing the invention. These Examples are in no way to be considered to limit the scope of the invention in any manner.

### Example 1

### Demonstration that Compounds of the Invention Prevent or Reduce Processes Associated with Restenosis

Compounds of the invention demonstrate activities consistent with the prevention or reduction of cellular mechanisms associated with restenosis, as shown herein below. Thus, the compounds, methods, and devices of the invention will be recognized by those of skill in the cardiovascular medicine arts as being effective to substantially prevent or reduce restenosis.

The effects of varying drug concentrations of paclitaxel, rapamycin, and a drug selected from the group consisting of epothilone D, 17-AAG, and 17-DMAG on growth characteristics for the same human smooth muscle cells (“SMCs” and endothelial cells (“ECs”) were compared under in-vitro experimental conditions as described hereinbelow.

Human aortic smooth muscle cells (“AoSMCs”) and human umbilical vein endothelial cells (“HUVECs”) were plated on 96-well culture plates at a density of about 10,000 cells per square centimeter (10,000 cells/cm²). The density was determined using growth curves determined by calculating the average absolute optical densities (“ODs”, defined as cellular OD—media only OD) for each plating concentration, for each day, and each cell type (AoSMC or HUVEC) over a five-day time period. The AoSMCs were purchased frozen from Clonetics/Biowhittaker/Cambrex (Item # CC-2571/Lot # 0F0222). The AoSMCs had company-determined culture characteristics on arrival: a total cell number of 917,500; cell viability: 95%; and a doubling time of between about. twenty-four and forty-eight hours. Pooled HUVECs were also purchased in frozen aliquot from Clonetics/Biowhittaker/Cambrex (Item # CC-2519/Lot # 1F0832). The company-determined culture characteristics on arrival were: a total Cell number of 560,000; cell viability of 83%; and a doubling time of between about eighteen and about forty-eight hours.

Prior to experiments, the AoSMCs and HUVECS were thawed and independently propagated through two- or three population doublings following Clonetics recommendations and standard cell culture techniques. Clonetics Growth Media and Reagents were used without alteration in all aspects of the study described herein unless otherwise noted. The details of the media and reagents can be found at the Cambrex World-Wide Web site. SMGM contained: 500 ml SMBM-2 basal media, 5% FBS, and all recommended singlequot growth supplements (provided with SMGM-2 bulletkit) ECGM contained: 500 ml EBM basal media, 2% FBS, and all recommended singlequot growth supplements (provided with EGM-bulletkit).

After initial plating all 96-well plates were placed in a standard 37° C., 5% CO₂ incubator. Conditions, including media, were not changed except as detailed below. For consecutive 24-hour time-periods, from Day 1 to Day 5, a single 96-well plate was removed from the incubator. For this plate, culture media was removed from all wells and replaced with 100 microliters (“μl”) of MTS reagent/media solution (see below) and the plate was then placed back in the incubator 3-4 hours later this plate was removed from the incubator and optical density data for each well was obtained using a 96-well ELISA reader.

Cell Preparation Source cells were selected at 70-80% confluency of the second or third population doubling since initial thaw. In order to synchronize cell cycle, source cells were changed from standard growth media to media containing 1% serum twenty-four hours prior to experiment (other growth factors were unchanged). On Day 0 of the experiment, source cells were removed from culture dishes by trypsinization (0.05×1 min.-2 min), quantified by hemacytometer after centrifuge (800 RPM×5 min.), and re-suspended in media to obtain a stock solution of about 25,000 cells/ml.

The drugs were dissolved in dimethylsulfoxide (“DMSO”) solvent to make stock solutions, which were then diluted serially in media to three study concentrations (10 nM; 100 nM; and 1,000 nM). The drugs at three concentration each, solvent without drug at three concentrations, and standard media were added independently to cells on the first day of the study only. Cells in two columns (16 wells) for each cell type on each day will not receive drug and serve as internal controls. Cellular viability and proliferation was assessed using the MTS assay for each cell type at each of the six time points. Rapamycin was purchased from Sigma Aldrich as a 1 mg powder (Item # R0395). Paclitaxel was purchased from Sigma Aldrich as a 5 mg powder (Item # T7191). Epothilone D, 17-AAG, and 17-DMAG were obtained using the methods and materials described above.

On Day 1, six hours after initial plating, the media was removed from all wells by vacuum suction; and with the appropriate growth media-drug solution for each of three drugs at three concentrations described above was added to the wells. For each cell type (AoSMC or HUVEC), and on each day, two 96-well plates were required to incorporate all three drugs (24 total plates). Standard wells contained only media and were used for optical density (“OD”) control in each individual plate during analysis. Control wells contained the plate specific cells without drug or solvent, and served as the control for all drug effects on a given day for a given cell type.

For consecutive 24 hour time-periods, from Day 0 to Day 5, four 96-well plates were removed from the incubator (two plates each for AoSMCs and HUVECs). For these plates, the culture media was removed from all wells and replaced with 100 μl of MTS reagent solution, which contained 20 μl of Promega CellTiter 96 Aqueous One reagent in 80 μl of cell appropriate growth media for each well. Promega recommendations for assay use were followed throughout including reagent administration under dark lighting 3-4 hours later these plates were removed from the incubator and optical density data for each well or each plate was obtained using a 96-well automated ELISA reader. Plates were read within 1.5 hrs of the same time each day. Additional details for the CellTiter 96 Aqueous One Assay are available online at the Promega World-Wide Web site.

Optical Densities for each column were averaged (n=8). Using the MTS assay steps detailed above, the “standard” wells contained only MTS reagent in media at the time of analysis. The average OD values for these “standard” columns were then subtracted from those column averages of drug treated cells, in order to obtain an absolute OD for drug treated cells. The average OD values for the “standard” columns were also subtracted from those columns containing control cells in order to obtain an absolute OD for control cells.

Average absolute OD s for cells at a given drug concentration were plotted for Days 0-5 for both AoSMCs and HUVECs. Average absolute ODs for AoSMC and HUVEC control cells were plotted on the same respective graphs for Days 0-5.

The results of the study are shown in FIGS. 1 and 2. From the figures, those of skill in the pharmacology and medicine arts will understand that epothilone D, 17-AAG, and 17-DMAG each shows dose-response characteristics consistent with utility to reduce or prevent restenosis. Moreover, those of such skill will also understand from the data presented that 17-AAG, and 17-DMAG each shows relative selectivity for SMCs over ECs. Thus, the present invention also provides treatment methods and compositions that are relatively selective for SMCs over ECs.

### Example 2

### Demonstration of Synergistic Effects with Anti-Inflammatory Compounds

Human aortic smooth muscle cells were obtained from Cambrex (Walkersville, Md.). The cells were maintained in SmGM-2 growth medium (Cambrex). Rapamycin, 17-AAG, and KOS-862 were obtained as described above or from commercial sources. The compounds were dissolved in dimethylsulfoxide (“DMSO”) to a concentration of 10 mM and stored at −20° C.

The cells were seeded in duplicate, in opaque-walled 96-well microtiter plates at a cell density of 3,000 cells per well and allowed to attach overnight. Serial dilutions of each drug were added, and the cells were incubated for 96 hours. The IC₅₀ values for the drugs was determined using the CellTiter-Glo Luminescent Cell Viability Assay (Promega, Madison, Wis.), which correlates with the number of live cells.

For the drug combination assays, the cells were seeded in duplicate in 96-well plates (3,000 cells/well). After an overnight incubation, the cells were treated with drug alone or a combination of the drug and rapamycin. Based on the IC₅₀ values of each individual drug, combined drug treatments were designed to provide constant ratios of the two drugs being tested for synergistic effect, i.e., at a concentration equivalent to the ratio of their individual IC₅₀ values. Three different treatment schedules were used: The cells were treated with rapamycin and 17-AAG; or rapamycin and KOS-862 simultaneously for 96 hours. Cell viability was determined by luminescent assay (Promega). Combination analysis was performed by using Calcusyn software (Biosoft, Cambridge, UK).

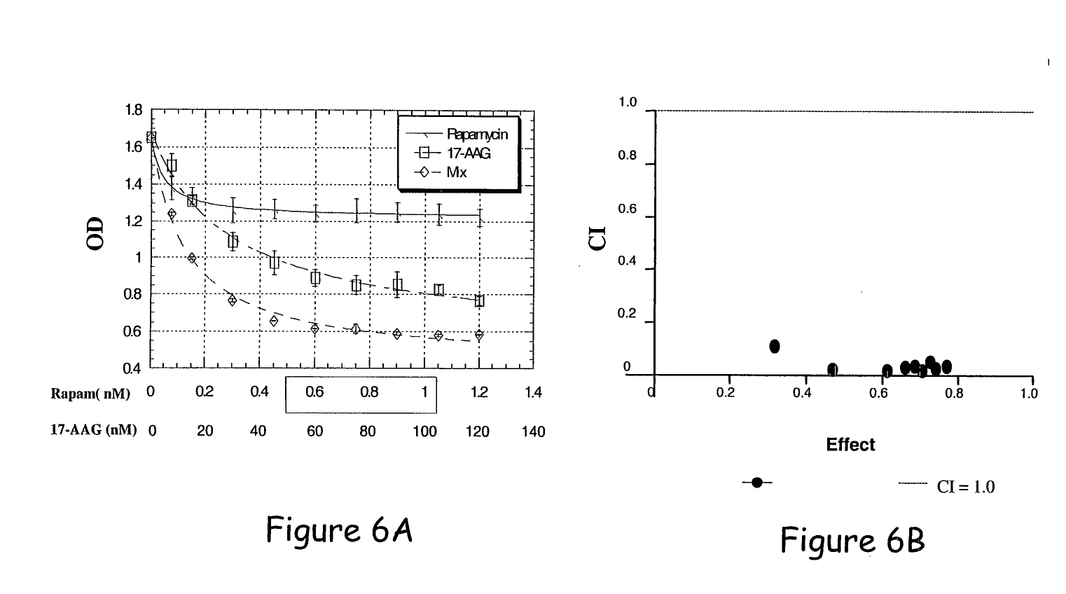

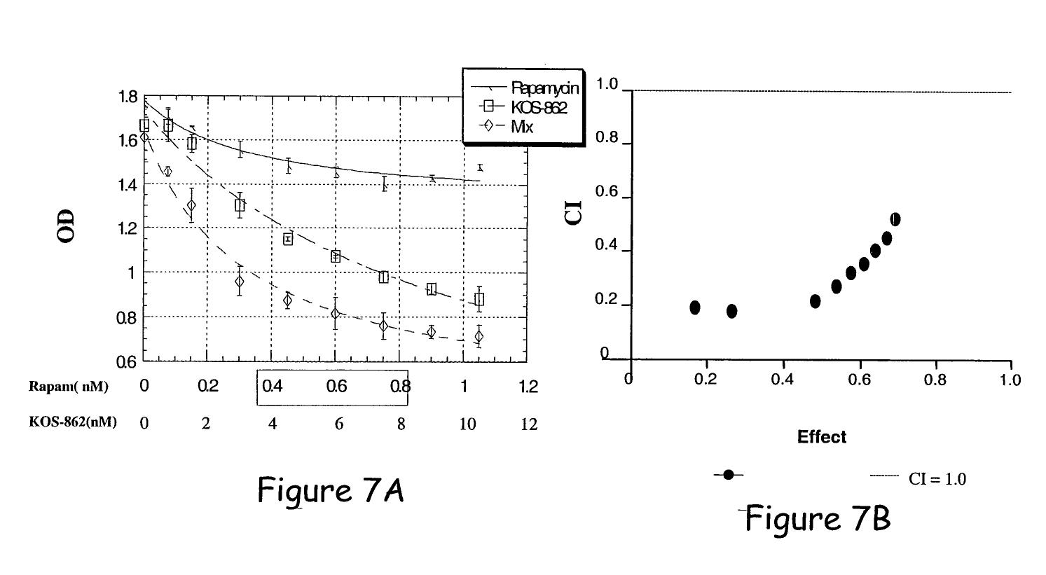

Each of the combinations of rapamycin and 17-AAG and rapamycin and KOS-862 was found to be synergistic as shown in FIGS. 4 and 5. Thus, each of these two combinations is likely to have better pharmacological effect in preventing or treating restenosis than the effect of either component alone. Synergy was also demonstrated using the procedure described above in DLD-1 cells (FIGS. 6 and 7).

### Example 3

### In Vitro Drug Elution of 17-AAG Matrixed PEA

The elution of 17-AAG from representative poly(ester-amide) coated stainless steel disks was determined by UV and HPLC methods. Stainless steel disks (0.71 cm²) were coated with polymer and 17-AAG by pipetting solutions of PEA-24-Bz and 17-AAG in absolute ethanol onto the disks and air drying overnight. In some cases, the coated disks were further topcoated with either PEA-24 or PEA-17, and then dried using the same techniques. Total drug loads of 50, 100, or 200 micrograms/cm² were used, with a drug load of either 10 or 20% (w/w) versus polymer. For elution, the disks were placed in a 15 mL plastic vial containing 1.5 mL of medium consisting of either chymotrypsin (0.4 mg/mL), phosphate buffered saline (PBS), fetal bovine serum (FBS), or human serum. The vials were incubated at 37° C., and the medium was sampled daily. Drug release was assayed by HPLC analysis of an aliquot pretreated by solid-phase extraction (see Example 4), or by the UV absorbance of the aliquot (200 uL), extrapolated from a calibration curve made from drug standards. The UV assay gave results consistent with 96% of theoretical.

The HPLC method entailed chromatography using a 250×4.6 mm 5 micron 100 A Zorbax Eclipse XDB C8 reversed-phase column with a 12.5×4.6 mm matching guard column. The mobile phases were A: 0.2% acetic acid in water, and B: 0.1% acetic acid in acetonitrile, flow rate 1 mL/min. A gradient elution was performed: 50% B for 2 minutes, then 9 minutes to 95% B, then isocratic at 95% B for 5 min, then back to 50% for 1 min and equilibrate for 4 min. 17-AAG was detected by UV at 330 nm.

The release data for 17-AAG into chymotrypsin medium demonstrated that 17-AAG is released at a sustained rate at least up to day 5, at which time the experiment terminated. Non-topcoated matrix released 17-AAG at a faster rate than topcoated matrix, with 56% total drug released over 5 days compared with 40% for the non-topcoated matrix.

The release data for 17-AAG into FBS medium demonstrated that 17-AAG is is released at a sustained rate at least up to day 4.5, at which time the experiment terminated. Non-topcoated matrix released 17-AAG at a faster rate than topcoated matrix, with 31% total drug released over 4.5 days compared with 21% for the non-topcoated matrix.

The effect of increased topcoating was studied using human serum medium. With a drug loading of 200 micrograms/cm², a 200 microgram topcoat gave 16% release of 17-AAG after 24 hours in human serum, whereas both a 400 microgram and a 600 microgram topcoate gave 6-7% release. Thus, doubling the topcoat has a significant effect on 17-AAG release, wheras tripling the topcoat has no further effect.

The solubility of 17-AAG in PBS is 60 micrograms/mL, and the IC₅₀ for endothelial cells is 350 nM. These studies suggest a drug loading of at least 200 micrograms of 17-AAG per stent with a 20-30% (w/w) drug/polymer formulation.

### Example 4

### Solid-Phase Extraction of Drugs from Serum

Drug aliquots from serum experiments were subjected to solid-phase extraction by loaded onto the top of 3 mL Isolute HM-N solid phase extraction columns (Argonaut; San Carlos, Calif.). After 5-10 minutes, the columns were eluted with 10-12 mL of chloroform:isopropanol (95/5 v/v) followed by 4 mL of ethyl acetate/isopropanol (95/5 v/v). The total eluate was concentrated, and the residues reconstituted and filtered through an 0.5 micron filter prior to HPLC analysis. Recovery of 17-AAG from the solid phase extraction was 88.7%. Recovery of epothilone D was 95-98%.

### Example 5

### Release Kinetics of 17-AAG from Coated Stents in Porcine Serum

Unmounted Metronic ‘Driver’ 18×3.5 mm stents were coated with MVPEA/17-AAG matrixed polymer material by spray coating. Three groups of stents were used in this study: (i) a low-dose group with 10% (w/w) drug load; these had 450 micrograms of polymer, 50 micrograms of 17-AAG, and 250 micrograms of topcoat; (ii) a high-dose group with 30% (w/w) drug load; these had 150 micrograms of polymer, 150 micrograms of 17-AAG, and 250 micrograms of topcoat; and (iii) a control group coated with polymer only (700 micrograms). Coated stents were sterilized by gamma irradiation.

Three stents from each group were placed aseptically into sterile glass vials and treated with 5 mL of sterile porcine serum at 37° C. with gentle agitation by shaking at 120 rpm. All 5 mL of serum was removed from each vial under sterile conditions at 0.5, 2, 4, 6, 12, and 24 hours, and then 2, 3, 5, 7, and 10 days. Fresh serum was added to the vials and incubation was continued after each time point. The time point aliquots were subjected to solid-phase extraction (see Example 4) prior to analysis by HPLC.

These studies demonstrated that 17-AAG was released into porcine serum along with an apparent metabolite.

### Example 6

### Release Kinetics of 17-AAG in Human Serum

Stainless steel disks (0.71 cm²) coated on one side with PEA-17-AAG having a drug loading of 100 ug/cm² (equal to 71 micrograms total 17-AAG) with either no topcoat or 210 ug, 420 ug, or 640 ug of a PEA topcoate (same as basecoat) were exposed to either human serum or chymotrypsin solution and 17-AAG was measured as described in Example 3. results are given in Table 1.

TABLE 1
Release kinetics of 17-AAG in human serum.
Percent of loaded drug found in serum as a function
of the amount of topcoat polymer over a 14-day period.

| Topcoat | Day 1 | Day 2 | Day 3 | Day 5 | Day 7 | Day 14 |
|---|---|---|---|---|---|---|
| 0 ug | 29% | 40% | 47% | 52% | 54% | 55% |
| 210 ug | 17% | 26% | 30% | 37% | 40% | 45% |
| 420 ug | 9% | 15% | 20% | 26% | 29% | 35% |
| 640 ug | 7% | 12% | 18% | 24% | 28% | 33% |

A disk coated with the PEA polymer exposed to chymotrypsin (0.4 mg/mL) showed increasing weight loss due to degradation of the polymer, with the PEA degradation by about 14% over 5 days and about 30% over 14 days. After 5 days in chymotrypsin solution, a drug-loaded disk released about 55%, indicating that drug release represents the combined effects of drug diffusion and matrix erosion.

The advantages and qualities of the invention will be apparent from the foregoing discussion. The present invention provides useful methods, compositions, devices, and drugs for reducing or preventing restenosis. Moreover, the invention provides useful methods, compositions, devices, and drugs for reducing or preventing restenosis that are selective for smooth muscle cells over endothelial cells. Thus, the present invention will be appreciated by those of skill in the pharmacology and medicine arts to provide treatments and prophylactics for restenosis that have reduced undesirable side effects compared to current restenosis treatment methodologies described herein. Furthermore, those of skill in the pharmacology, medicine, and medical device arts will understand that many alternative embodiments of the invention not explicitly described herein are nevertheless encompassed by the present invention. Examples of such alternative embodiments include, but are not limited to, particular combinations of polymers for drug delivery, particular stents, and particular methods of drug delivery.

All references cited herein are hereby incorporated by reference for all purposes.

1. A medical device configured to deliver one or more drugs to a blood vessel to reduce the degree or substantially prevent the occurrence of restenosis in the blood vessel, said drugs selected from the group consisting of epothilone D, 17-allylamino-17-desmethoxygeldanamycin, 17-[2-(dimethylamino)-ethylamino]-17-desmethoxygeldanamycin, 17-[2-(dimethylamino)-ethylamino]-17-desmethoxy-11-O-methylgeldanamycin, and 17-azetidinyl-17-desmethoxygeldanamycin.

2. The medical device of claim 1 wherein the medical device is a stent or polymer wrapper.

3. The medical device of claim 2 wherein the drug is epothilone D.

4. The medical device of claim 2 wherein the drug is selected from the group consisting of 17-allylamino-17-desmethoxygeldanamycin (“17-AAG”), 17-[2-(dimethylamino)ethylamino]-17-desmethoxygeldanamycin (“17-DMAG”), 17-[2-(dimethylamino)ethylamino]-17-desmethoxy-11-O-methylgeldanamycin, and 17-azetidinyl-17-desmethoxygeldanamycin.

5. A medical device configured to deliver a first and a second drug to a blood vessel to reduce the degree or substantially prevent the occurrence of restenosis in the blood vessel, said first and second drugs selected from the group consisting of epothilone D, rapamycin, a rapamycin analog, 17-allylamino-17-desmethoxygeldanamycin, 17-[2-(dimethylamino)ethylamino]-17-desmethoxy-geldanamycin, 17-[2-(dimethylamino)-ethylamino]-17-desmethoxy-11-O-methylgeldanamycin, and 17-azetidinyl-17-desmethoxygeldanamycin; wherein said first and second drugs show synergistic cytotoxic activity.

6. The medical device of claim 5 wherein the medical device is a stent or polymer wrapper

7. The medical device of claim 6 wherein the first drug is selected from the group consisting of epothilone D, 17-allylamino-17-desmethoxygeldanamycin, 17-[2-(dimethylamino)ethylamino]-17-desmethoxy-geldanamycin, 17-[2-(dimethylamino)-ethylamino]-17-desmethoxy-11-O-methylgeldanamycin, and 17-azetidinyl-17-desmethoxygeldanamycin; and the second drug is rapamycin or a rapamycin analog.

8. The medical device of claim 6 wherein the first drug is epothilone D and the second drug is rapamycin or a rapamycin analog.

9. The medical device of claim 6 wherein the first drug is selected from the group consisting of 17-allylamino-17-desmethoxygeldanamycin, 17-[2-(dimethylamino)ethylamino]-17-desmethoxy-geldanamycin, 17-[2-(dimethylamino)-ethylamino]-17-desmethoxy-11-O-methylgeldanamycin, and 17-azetidinyl-17-desmethoxygeldanamycin; and the second drug is rapamycin or a rapamycin analog.

10. The medical device of claim 5 wherein the drugs showing synergistic cytotoxic activity are 17-AAG and rapamycin.

11. The medical device of claim 5 wherein the drugs showing synergistic cytotoxic activity are epothilone D and rapamycin.

12. The medical device of claim 5 wherein the drugs showing synergistic cytotoxic activity are 17-[2-(dimethylamino)ethyl]-17-desmethoxygeldanamycin and rapamycin.

13. A composition comprising a polymer and one or more cytotoxic drugs selected from the group consisting of epothilone D, 17-allylamino-17-desmethoxygeldanamycin (“17-AAG”), 17-[2-(dimethylamino)ethylamino]-17-desmethoxygeldanamycin (“17-DMAG”), 17-[2-(dimethylamino)ethylamino]-17-desmethoxy-11-O-methylgeldanamycin, and 17-azetidinyl-17-desmethoxygeldanamycin.

14. A composition comprising a polymer and a first and second drug, said first and second drugs selected from the group consisting of epothilone D, rapamycin, a rapamycin analog, 17-allylamino-17-desmethoxygeldanamycin, 17-[2-(dimethylamino)ethylamino]-17-desmethoxy-geldanamycin, 17-[2-(dimethylamino)ethylamino]-17-desmethoxy-11-O-methylgeldanamycin, and 17-azetidinyl-17-desmethoxygeldanamycin; wherein said first and second drugs show synergistic cytotoxic activity.

15. The composition of claim 14 wherein the first drug is epothilone D and the second drug is rapamycin or a rapamycin analog.

16. The composition of claim 14 wherein the first drug is selected from the group consisting of 17-allylamino-17-desmethoxygeldanamycin, 17-[2-(dimethylamino)ethylamino]-17-desmethoxy-geldanamycin, 17-[2-(dimethylamino)-ethylamino]-17-desmethoxy-11-O-methylgeldanamycin, and 17-azetidinyl-17-desmethoxygeldanamycin; and the second drug is rapamycin or a rapamycin analog.

17. The composition of claim 14 wherein the first drug is 17-allylamino-17-desmethoxygeldanamycin and the second drug is rapamycin or a rapamycin analog.

18. The composition of claim 14 wherein the first drug is 17-[2-(dimethylamino)ethylamino]-17-desmethoxy-geldanamycin and the second drug is rapamycin or a rapamycin analog.

19. The composition of claim 13 wherein the polymer is selected from the group consisting of a polylactide, a photo-curable poly(ester-amide), and a polyurethane.

20. A method to reduce the degree or substantially prevent restenosis in a blood vessel, comprising delivering a composition of claim 13 to a blood vessel requiring treatment for, or prevention of, restenosis, in an amount sufficient to substantially reduce, or substantially prevent, restenosis in such blood vessel.
